(19) United States
(12) Patent Application Publication
(10) Pub. No.: US 2004/0204503 A1
(43) Pub. Date: Oct. 14, 2004

(54) Method and apparatus for storage and transportation of hydrogen

(76) Inventor: James H. Beyer, Ann Arbor, MI (US)

Correspondence Address: MITCHELL M. MUSIAL II, PLLC, 6960 ABBOTT TERRACE, WEST BLOOMFIELD, MI, 48323, US

(21) Appl. No.: 10/779,098

(22) Filed: Feb. 14, 2004

Related U.S. Application Data
(60) Provisional application No. 60/462,234, filed on Apr. 11, 2003.

(51) Int. Cl. C07C 27/06

(52) U.S. Cl. 518/703

(57) ABSTRACT

Disclosed herein is an apparatus and method for storing and transporting hydrogen by employing carbon dioxide as a storage medium. An electrolyzer uses energy from renewable sources to provide hydrogen by dissociating water. A reactor forms a product by reacting hydrogen and carbon dioxide. The product is transported to a consumption location or the storage location. A storage device may be employed to store retained carbon dioxide produced when the product is consumed. Retained carbon dioxide is transported to the reactor location to be reacted with the hydrogen provided from a hydrogen source. As such, a carbon dioxide circuit is used to efficiently transport and store hydrogen.

### CROSS-REFERENCE TO RELATED APPLICATIONS

This application claims the benefit of and priority to prior filed co-pending U.S. provisional application serial No. 60/462,234, filed Apr. 11, 2003, the disclosure of which is incorporated herein by reference.

### FIELD OF THE INVENTION

The present invention relates to a method and apparatus for storing hydrogen. More specifically, the invention relates to storing and transporting hydrogen by employing carbon dioxide as a storage medium.

### BACKGROUND OF THE INVINTION

Fossil fuels such as methane (CH₄) provide energy but at the expense of producing CO₂ emissions. Renewable energy sources such as solar power and wind provide intermittent energy, including electrical energy, that is difficult to store, and as such, is not easily useable to supplement energy demands. However, energy from renewable sources can be used to easily produce hydrogen by electrolyzing water. Furthermore, hydrogen can be obtained by reforming hydrocarbon products such as methane or diesel fuel. Hydrogen can also be produced by nuclear power, electrolysis or steam electrolysis (making use of waste heat).

Accordingly, hydrogen storage has been an area of intense research because hydrogen is abundant and a superior fuel for many applications. Hydrogen may be used to produce electricity by employing a device such as a fuel cell, which produces only water vapor as a byproduct. Hydrogen is a favored fuel because fuel cells are more efficient at using the energy content of hydrogen than internal combustion engines are at using the energy content of diesel fuel or gasoline (roughly 40% versus 30% energy usage). However, fuel cells are not mature technologies. Furthermore, there are concerns with transporting hydrogen.

There are difficulties involved with hydrogen storage. Although hydrogen has very high energy capacity per unit mass, it has a very low density, even in liquid form, and consequently is very bulky as a fuel. Storage is a major concern especially for mobile applications, as the tank must be on board the vehicle. A liter of hydrogen, compressed to 400 times standard pressure, contains only the energy value of 0.24 liters of gasoline or diesel, even taking into account the improved efficiency of fuel cells. A liter of liquefied hydrogen has a higher energy value than the compressed hydrogen referenced above is a bit better, equal to about 0.475 liter of gasoline. Hydrogen must be made very cold to liquefy, about −423 F/−253C, which requires energy input. Tanks that are designed to retain liquid hydrogen are also expensive. Hydrogen can be compressed to 660 times atmospheric pressure or more, but this requires additional energy, and these tanks become very expensive to build.

Because of the problems of storing hydrogen directly, other fuel sources that can be stored more easily are sought. These compounds are then processed, or reformed, to release the hydrogen for use. Compounds such as these include methanol, ethanol, methane, and even gasoline, can be reformed to release hydrogen. One problem with this method is that carbon dioxide is released, which means it is not a useable strategy for a zero emission vehicle (ZEV). Furthermore, these fuels do not leverage renewable energy sources.

Other compounds, such as hydrides can be used to hold hydrogen. Some metal hydrides can be heated to release their hydrogen and then later must be restored or “recharged” during a refueling process. Other hydrides, such as sodium borohydride, release hydrogen when exposed to water but leave a residue on the storage material, which must be processed to be recharged.

A final category for hydrogen storage is to use new or exotic materials, including nanotubes, to store hydrogen. The new materials have an immense array of tiny surfaces to which hydrogen can attach and then release, producing a storage mechanism. However, this technology is not yet mature or proven to work effectively.

Thus, it is desirable to provide a cost effective means of storing hydrogen without having to expend a significant amount of energy to compress or liquefying the gas. Furthermore, it is desirable to provide a cost effective means of transporting hydrogen. It is further desirable to provide a method of effectively harnessing renewable energy sources. Lastly, it would be advantageous to provide an energy storage and transportation system that precludes net carbon dioxide emissions during energy consumption.

### SUMMARY OF THE INVENTION

An apparatus for transporting hydrogen comprises a hydrogen source and a carbon dioxide source. A reactor is in communication with the hydrogen source and the carbon dioxide source for causing hydrogen to react with carbon dioxide to form a product selected from the group consisting of a hydrocarbon and an oxygenated hydrocarbon. A conduit is in communication with the reactor for transporting the product to either a consumption location or storage location. A conduit is in communication with a consumption location for transporting carbon dioxide to either a reactor location or storage location.

A method of transporting hydrogen comprises the steps of providing a source of hydrogen and a source of carbon dioxide. The hydrogen and the carbon dioxide are conducted to a reactor. Hydrogen is reacted with carbon dioxide to form a product selected from the group comprising a hydrocarbon and an oxygenated hydrocarbon. The product is transported to either a consumption location or storage location. Carbon dioxide is transported from a consumption location to one of a reactor location or storage location.

An apparatus for storing hydrogen by using carbon dioxide as a storage medium comprises a hydrogen source and a carbon dioxide source. A reactor is in communication with the hydrogen source and the carbon dioxide source for causing hydrogen to react with carbon dioxide to form a product selected from the group consisting of a hydrocarbon and an oxygenated hydrocarbon. A storage device is in communication with the reactor for storing the product containing hydrogen.

A method of storing hydrogen by using carbon dioxide as a storage medium comprises the steps of providing an amount of hydrogen and an amount of carbon dioxide. The hydrogen and the carbon dioxide are conducted to a reactor to form a product selected from the group consisting of a hydrocarbon and an oxygenated hydrocarbon. The product containing hydrogen is stored.

Further objects, features and advantages of the present invention will become apparent to those skilled in the art from analysis of the following written description, the accompanying drawings and the appended claims.

### BRIEF DESCRIPTION OF THE DRAWINGS

FIG. 1 is a schematic of an energy usage system according to the current state of the art where methane is used as a fuel and carbon dioxide is released into the atmosphere.

FIG. 2 is a schematic of an energy usage system according to the current state of the art where renewable energy sources are not included as part of a fuel source and carbon dioxide is released into the atmosphere.

FIG. 3 is a schematic of an energy usage system where natural and renewable energy are converted into hydrogen for transportation, revealing the release of carbon dioxide into the atmosphere when natural gas is converted into hydrogen.

FIG. 4 is a diagram of a methane/carbon dioxide circuit for transporting hydrogen from a point “A” to a point “B” compared to transporting hydrogen from a point “A” to a point “B”.

FIG. 5 is a diagram of a carbon dioxide circuit for transporting hydrogen from an energy production location to an energy use location.

FIG. 6 is a schematic of an operative element according to the principles of the present invention, revealing a Sabatier reactor in communication with a hydrogen source and carbon dioxide source to form a product, specifically, methane.

FIG. 7 is a schematic of an apparatus according to the principles of the present invention.

FIG. 8a is a schematic of an apparatus according to the principles of the present invention, revealing an embodiment for hydrogen transportation.

FIG. 8b is a schematic of an alternative apparatus according to the principles of the present invention, revealing an embodiment for hydrogen storage.

FIG. 8c is a schematic of an alternative apparatus according to the principles of the present invention, revealing an embodiment for carbon dioxide storage.

### DESCRIPTION OF THE PREFERRED EMBODIMENT

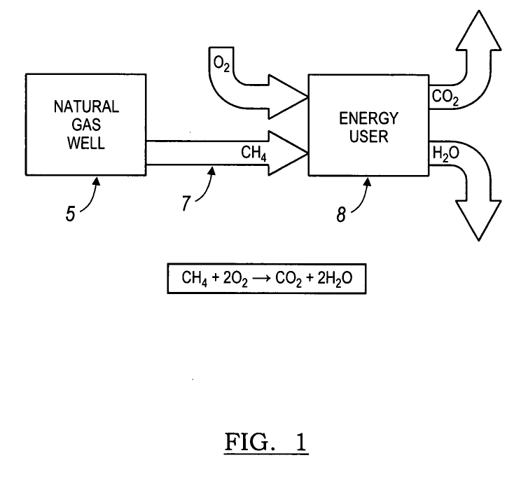

With initial reference to FIG. 1, a schematic of an energy usage system according to the current state of the art is shown. A natural gas source 5, specifically a gas well, is in communication with a gas conduit 7 to transport natural gas to an energy user at a consumption location 8. The energy user will consume the natural gas by combustion of the natural gas with oxygen to generate heat and produce carbon dioxide and water as byproducts, assuming the combustion is ideal.

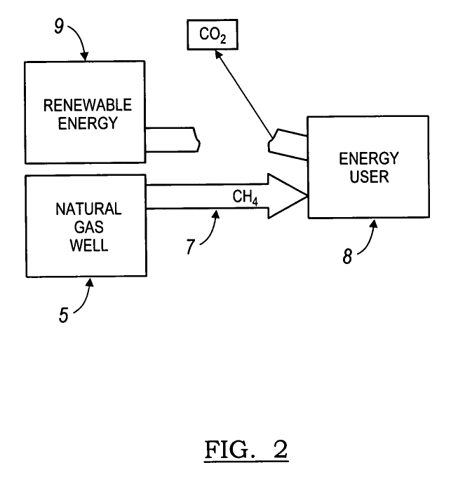

Referring now to FIG. 2, a schematic of the energy usage system of FIG. 1 is shown, further comprising a renewable energy source 9, according to the current state of the art. Renewable energy sources are difficult to employ in order to supplement energy demand because the energy from renewable sources, such as wind and solar energy, are not consistent. Renewable energy sources can easily produce electricity, but can only sporadically reduce fixed loads from traditional electrical power sources. Electricity from renewable energy sources is also difficult to store in large quantities. Furthermore, electricity becomes inefficient to transmit via high voltage power lines more than a few hundred miles. Accordingly, renewable energy source 9 is shown not connected to the energy user at consumption location 8. Meanwhile, carbon dioxide is being released into the atmosphere, which is suspected to be a cause of global warming.

The prior art reveals that the world's energy system has significant shortcomings, including the absence of an acceptable use of renewable energy and carbon dioxide (CO₂) emissions which threaten the world with global warming.

A solution is desired to this problem that makes energy from renewable sources 9 accessible, stable in price and quantity, and low cost. To address the role of renewable sources, and to avoid CO₂ emissions, a solution referred to as the hydrogen economy is considered.

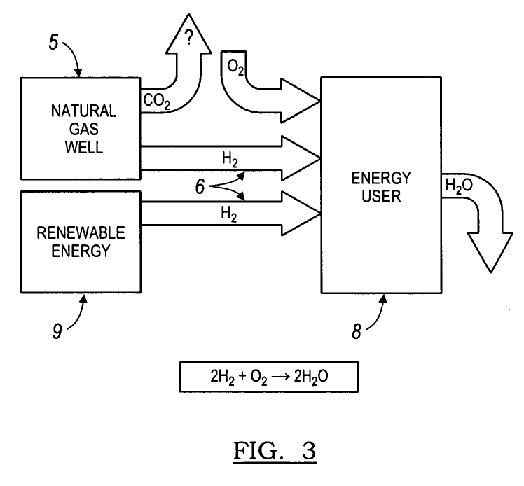

Referring now to FIG. 3, a schematic of an energy usage system is shown where natural gas and renewable energy are converted into hydrogen for transportation. Energy from renewable energy source 9 is converted to electrical energy, which is provided to an electrolyzer (not shown) to dissociate water into hydrogen and oxygen. A hydrogen conduit 7 connects a renewable energy source 9 to a consumption location 8 for transportation of hydrogen gas. A reformer (not shown) may be employed to reform natural gas from a natural gas source 5 to hydrogen and carbon dioxide. A hydrogen conduit 7 connects a natural gas source 5 to the consumption location 8 for transporting hydrogen.

In the present embodiment, renewable sources use electrical power to produce hydrogen by the electrolysis of water. The hydrogen is then conducted to consumers as a substitute for hydrocarbon fuel. Since the product from combustion of hydrogen is water, no carbon dioxide is produced. Additionally, fossil fuels are reformed into hydrogen as well to meet energy demands. The byproduct of the reforming step is carbon dioxide. The carbon dioxide from the reforming step would have to be captured or simply vented. If the carbon dioxide is vented, the hydrogen economy does not avoid carbon dioxide emissions; the carbon dioxide emissions are simply deferred.

While the hydrogen economy setting seems to work in theory, there are some concerns with it. First, our entire infrastructure has to change to make use of hydrogen as a fuel. Second, hydrogen is bulky and difficult to transport or to store. Therefore, renewable sources would be accessible only after this hydrogen infrastructure is in place.

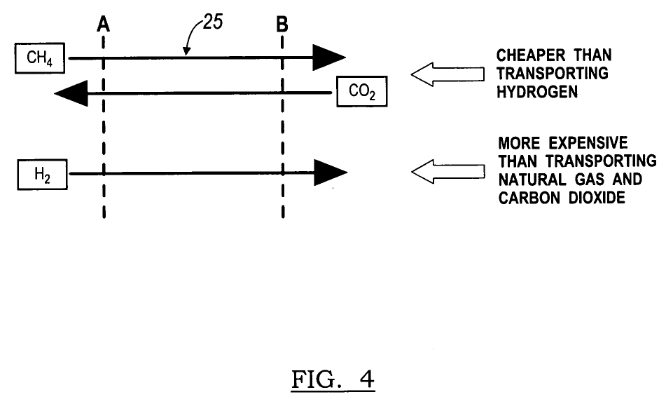

Referring now to FIG. 4, a diagram of a carbon dioxide circuit 25 for transporting hydrogen is shown. The circuit 25 transports hydrogen from a point “A” to a point “B” by reacting the hydrogen with carbon dioxide to form a product, which in the preferred embodiment is methane. The diagram of FIG. 4 shows that by employing carbon dioxide as a storage medium, hydrogen may be transported from point “A” to point “B”, returning the carbon dioxide to be “recharged” at point “A”. The premise of the present invention is that it is more efficient to transport a product of a reaction between carbon dioxide and hydrogen, including a hydrocarbon, such as methane, or oxygenated hydrocarbon, such as methanol, and the carbon dioxide to be reacted with the hydrogen, than it is to transport hydrogen from a point “A” to a point “B”.

Between any two points A and B, it is cheaper to transport a mole of methane from A to B and a mole of carbon dioxide from B to A, than it is to simply transport hydrogen, of equal energy content, from A to B. For example, one mole of carbon dioxide reacts with four moles of hydrogen to produce one mole of methane and two moles of water.

Although the present assertion seems counterintuitive, consider that the two major methods of moving hydrogen are either by a storage tank, or by a pipeline. In the case of the storage tank, it is well known that compressed methane is a more dense energy carrier than hydrogen. Therefore, a given tank of methane will hold more energy than hydrogen at the same pressure. After discharging, the hydrogen tank must be returned empty to the source for refueling. The methane tank is instead filled with carbon dioxide on its return trip. The carbon dioxide is transported with the returned container.

In the case of the pipeline, methane is more than twice as dense, from an energy per unit volume standpoint, than a given volume of hydrogen at the same pressure. Given two pipelines, the first containing methane, and the second containing carbon dioxide, moving in the opposite direction, can carry more energy than a single hydrogen pipeline that is more than twice the size of a methane pipeline, containing only hydrogen at the same pressure. Since methane is more than twice as dense energetically than hydrogen, even the combined compression costs of both the methane and carbon dioxide gases are less than hydrogen alone.

This assertion can be more formally supported by considering the following. Hydrogen has an energy capacity of 33.90 kilowatt-hours/kilogram. Methane has a capacity of 13.44 kilowatt-hours/kilogram. A mole of hydrogen is 2 grams, yielding 500 moles of hydrogen per kilogram. A mole of methane is 16 grams, yielding 62.5 moles of methane per kilogram. On a mole basis, the energy content of hydrogen is 0.0678 kilowatt-hours/per mole. Methane, however, has a capacity of 0.215 kilowatt-hours per mole. The combustion of one mole of methane produces one mole of carbon dioxide. Accounting for the carbon dioxide, the energy capacity of methane/carbon dioxide is still 0.1075 kilowatt-hours/mole. This is more than 58% greater than hydrogen. Energy content per mole is important because the work required to compress a gas is dependent on the number of moles of the gas, not its weight. Not wishing to be bound by theory, it is believed that methane and carbon dioxide require less energy to compress than hydrogen because each has a higher critical temperature and lower critical pressure than hydrogen does.

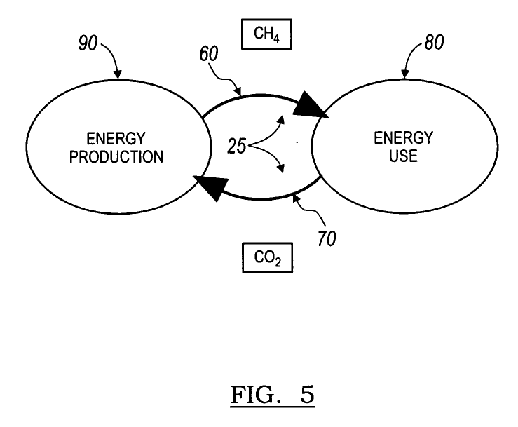

Referring now to FIG. 5, a diagram of a carbon dioxide circuit 25 for transporting hydrogen from a reactor location 90 to a consumption location 80 is shown. A product conduit 60 is in communication with the reactor location 90 and consumption location 80 for transporting a product, which in the present embodiment is methane, from the reactor location 90. A carbon dioxide conduit 70 is in communication with the consumption location 80 and the reactor location 90 for transporting carbon dioxide from the consumption location 80.

Since it is cheaper to transport methane and carbon dioxide in a circuit, the hydrogen economy plan may be modified. Instead of employing a single pipe of hydrogen, substitute two pipes for the hydrogen pipe, one of methane going from energy production to energy use, and the other carbon dioxide going from energy user to energy production.

At the energy consumption location 80, rather than venting carbon dioxide to the atmosphere, conduit 70 transports carbon dioxide back to the reactor location 90. Users of large quantities of energy retain CO₂ regularly, therefore, the ability to retain CO₂ is not a concern; the concern has been disposing of the retained CO₂. Accordingly, any method known in the art for sequestering CO₂ may be employed. The present invention that provides an apparatus and method for storage and transportation of hydrogen also provides a need for CO₂.

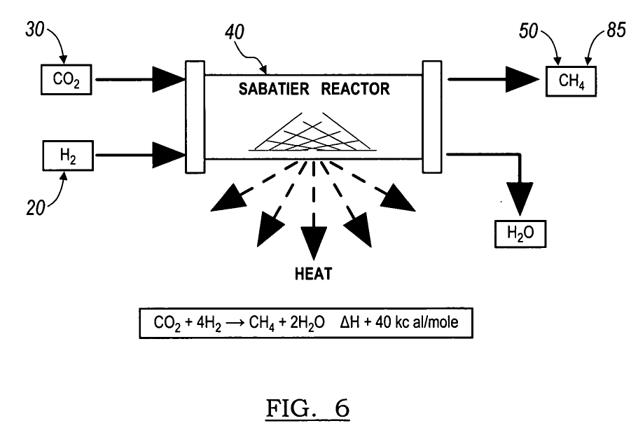

Referring now to FIG. 6, a schematic of an operative element according to the principles of the present invention is shown. A reactor 40, which in the present embodiment is a Sabatier reactor, is in communication with a hydrogen source 20 and carbon dioxide source 30 to form a product 50, specifically, methane. Although a Sabatier reactor is disclosed herein, those skilled in the art will immediately recognize that any suitable substitute may be employed, including, but not limited to, photo-electrolyzing devices.

Production of hydrogen for the present invention is accomplished by an electrolyzer, which dissociates water by introducing an electrical current, forming hydrogen and oxygen, as a byproduct. For example, 9 kilograms of water will produce 8 kilograms of oxygen and 1 kilogram of hydrogen, as demonstrated by the following chemical reaction:

4H₂O→2O₂+4H₂

A Sabatier reactor, in simple terms, is typically a metal tube containing a catalyst, such as nickel or ruthenium. The hydrogen reacts exothermically with the retained carbon dioxide to produce methane and water. As a Sabatier reactor is exothermic, energy is lost in the system. When hydrogen is reacted with carbon dioxide, about 79% of the energy content of hydrogen is stored as methane, with the balance released as heat. Some of the low-grade heat released by the Sabatier reactor may be employed for other uses. For example, 5.5 kilograms of carbon dioxide reacted with 1 kilograms of hydrogen will produce 2 kilograms of methane and 4.5 kilograms of water, as demonstrated by the following chemical reaction:

CO₂+4H₂→CH₄+2H₂O

Overall, a renewable energy site may be 60-80% efficient in producing methane according to the principles disclosed herein by using CO₂ as a carrier, versus 70-90% efficiency in producing hydrogen alone.

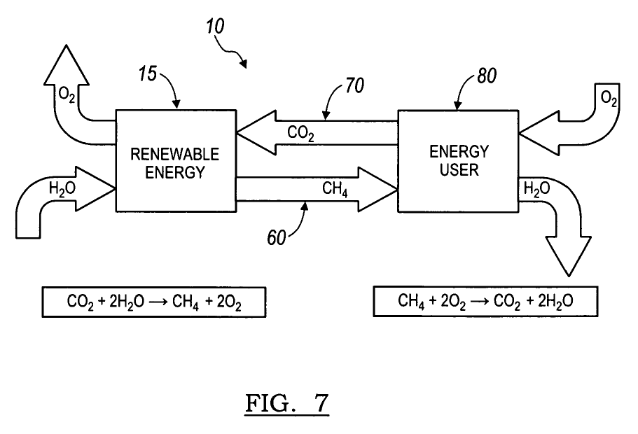

Referring now to FIG. 7, a schematic of an apparatus according to the principles of the present invention is shown. Energy from a renewable energy source 15 is used to convert water to hydrogen and oxygen. As such, a renewable energy source 15 functions as a source of hydrogen by dissociating water. A conduit 70 is in communication with a reactor (not shown in this figure) for transporting carbon dioxide to the reactor from a carbon dioxide source. The reactor causes hydrogen to react with the carbon dioxide to form a product, which in the present embodiment is methane.

A conduit 60 transports the product to a consumption location 80. At the consumption location 80 the product is consumed in the presence of oxygen yielding water and carbon dioxide as byproducts. In the present embodiment, the consumption location 80 is a source of carbon dioxide, which is used by the reactor to convert hydrogen to a product, such as a hydrocarbon or an oxygenated hydrocarbon. As such, carbon dioxide is employed as a storage medium for hydrogen. In addition, a renewable energy source may provide methane as a fuel source rather than low quality, intermittent electrical energy. Methane, in the form of natural gas, has long been economically transported in pipelines thousands of miles long, one of which extends from Louisiana to Michigan. Alternatively, electricity is uneconomical to transmit more than a few hundred miles due to resistance losses of the wires. Furthermore, carbon dioxide is not released into the environment, which provides an environmental benefit.

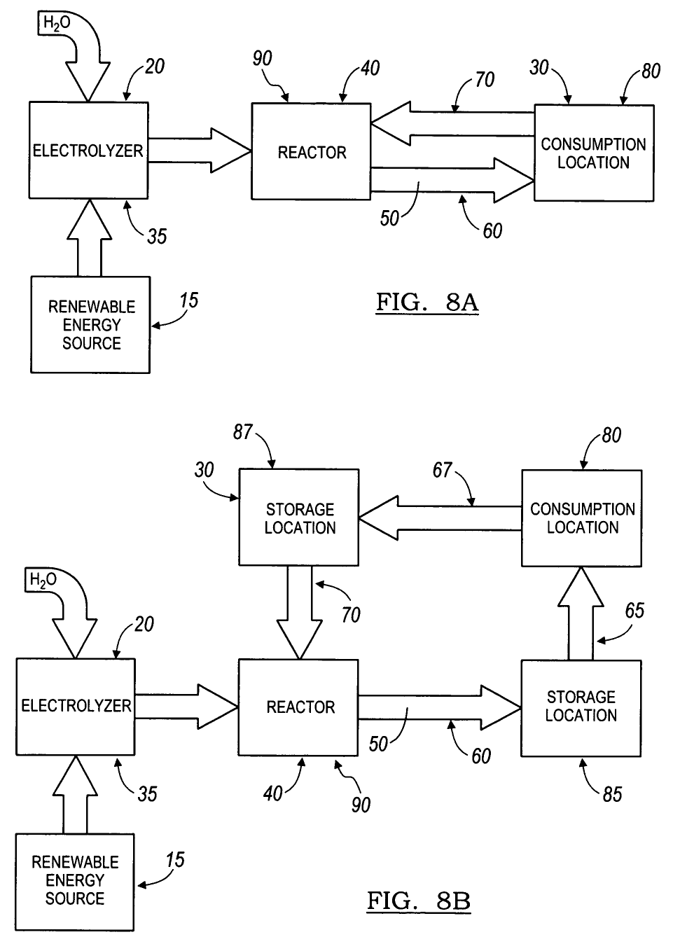

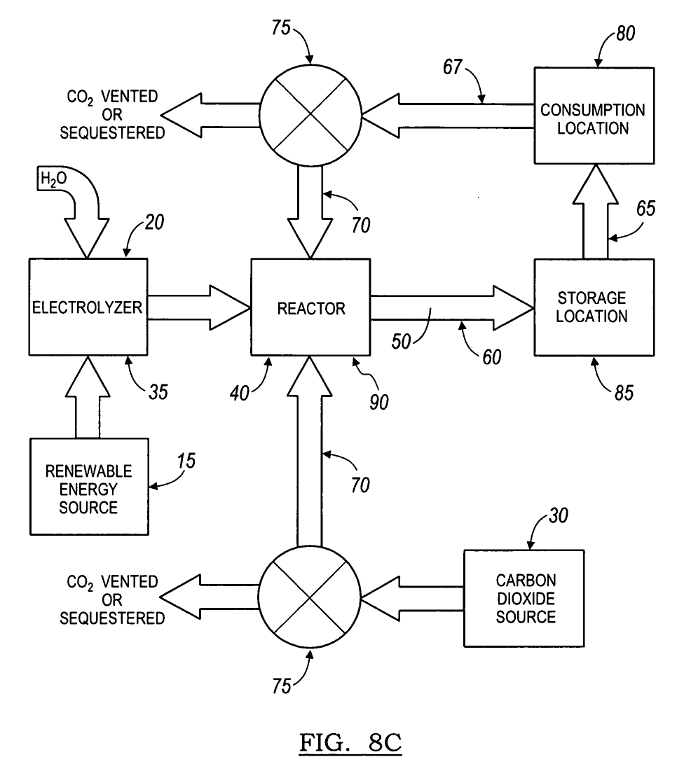

Referring now to FIG. 8a, a schematic of an apparatus according to the principles of the present invention is shown, revealing an embodiment for hydrogen transportation. An electrolyzer 35 receives energy from a renewable energy source 15 and water to produce hydrogen. As such, electrolyzer 35 is a hydrogen source which is in communication with reactor 40. Reactor 40 is located at a reactor location 90, which may be any suitable location. A carbon dioxide source 30 provides carbon dioxide to the reactor 40. The reactor 40 causes the hydrogen to react with the carbon dioxide to form a product 50 selected from the group consisting of a hydrocarbon and an oxygenated hydrocarbon. A product conduit 60 is in communication with the reactor 40 for transporting the product 50 to a consumption location 80. A carbon dioxide conduit 70 is in communication with the consumption location 80 for transporting carbon dioxide to the reactor location 90.

Referring now also to FIG. 8b, a schematic of an apparatus according to the principles of the present invention is shown, revealing an embodiment for hydrogen storage. The electrolyzer 35 receives energy from the renewable energy source 15 to provide a source of hydrogen to reactor 40. The reactor 40 combines hydrogen and carbon dioxide to form a product 50 for storage in a tank (not shown) or any suitable device provided at a storage location 85. A product conduit 60 may be in communication with the reactor 40 to transport the product 50 from the reactor location 90 to the storage location 85 for future use. When an energy demand requires the product 50 for consumption, a product conduit 65 may be employed to conduct the product 50 to consumption location 80.

Once the product 50 is consumed, carbon dioxide from the consumption location 80 is conducted to a storage location 87 for storage in a tank (not shown), or any suitable device, provided at a storage location 87. Storage location 87 may also serve as a carbon dioxide source 30.

Referring now also to FIG. 8c, a schematic of an apparatus according to the principles of the present invention is shown, revealing an alternative embodiment for carbon dioxide storage. The electrolyzer 35 receives energy from the renewable energy source 15 to provide a source of hydrogen to reactor 40. The reactor 40 combines hydrogen and carbon dioxide to form a product 50 for storage in a tank (not shown) or any suitable device provided at a storage location 85. A product conduit 60 may be in communication with the reactor 40 to transport the product 50 from the reactor location 90 to the storage location 85 for future use. When an energy demand requires the product 50 for consumption a product conduit 65 may be employed to conduct the product 50 to consumption location 80.

Once the product 50 is consumed, carbon dioxide from the consumption location 80 may be conducted back to the reactor 40 or vented or sequestered, depending on the state of a control valve 75. Alternatively, carbon dioxide may be extracted from a carbon dioxide source 30, such as a coal fired electricity generator, an underground well or ethanol production facility and directed by a control valve 75 to a reactor 40 or sequestered or vented. It should be noted that any suitable technology know in the art for storing and extracting carbon dioxide may be employed in the present invention.

Accordingly, the present invention incorporates carbon dioxide as a “hydrogen carrier”, which circulates in the system of the present invention rather than being released into the atmosphere. The invention can also allow for carbon dioxide to be released into the atmosphere where carbon dioxide capture may be expensive (such as in a vehicle) and be replaced by carbon dioxide which can be more easily retained from a non-consumption location, such as from an ethanol production facility.

The present invention can be adapted to motor vehicles, which would run on the product formed by the present invention rather than hydrogen. In order to accomplish this adaptation, the carbon dioxide from combustion could be retained during use. Adapting the present intention to order vehicles could be achieved by providing a plurality of tanks, where at least one tank contains the product formed by the present invention, and at least another for receiving carbon dioxide.

Refueling could be accomplished by evacuating the tank containing CO₂ and refilling the evacuated tank with methane. The evacuated CO₂ would then would be stored or provided to a reactor for production. The storage and transportation system of the present invention solves the problems regarding vehicle fuel cells, storing liquefied hydrogen, and emissions.

A vehicle, being a first consumption location, could also vent carbon dioxide to the atmosphere, as long as it was replaced with another source, such as from ethanol production, being a non-consumption location, or another consumption location, being a second consumption location. A vehicle may also be able to partially retain its carbon dioxide produced with the resulting partial benefit of returning the carbon dioxide.

It should be noted that although methane is referenced in the preferred embodiment of the present invention as the product formed by reacting hydrogen and carbon dioxide, any hydrocarbon or oxygenated hydrocarbon may be substituted for methane.

Not wishing to be bound by theory, it is believed that more complex hydrocarbons, such as ethane, propane, and butane may be preferred products for hydrogen storage as it might be easier to store complex hydrocarbons more densely than methane, in the same way methane is stored more densely than hydrogen.

Although current infrastructure supports natural gas for use, the tank storage infrastructure is fairly advanced for propane, C₃H₈. Ethane, C₂H₆, seems to be more difficult to store than propane, and more expensive to produce than methane.

It is believed that forming octane C₈H₁₈ from electrolyzed hydrogen would be cost prohibitive, but is deemed to be within the scope of the present invention. Although alcohols are believed to be inferior to the alkane series, CH₄, C₂H₆, C₃H₈, production of oxygenated hydrocarbons, including alcohols, are also deemed to be within the scope of the present invention.

Ethylene, C₂H₄, may also be a product within the scope of the present invention. Since ethylene has a double carbon bond, it is an alkene. Either liquefied ethylene or ethane C₂H₆ can be stored at about 1200 psi at room temperature, compared with 7500 psi for methane. Ethylene can also be reformed, using a Sabatier reactor for example, into ethane or propane which can be stored at room temperature at 250 psi.

Carbon dioxide is heavier than methane, but it liquefies under compression at much lower pressure. Carbon dioxide needs to be compressed to about 1000 psi to be retained as a liquid at room temperature. Methane requires a pressure of 5000-7500 psi at room temperature for high density storage. Hydrogen cannot be stored as a liquid at room temperature.

Although renewable energy sources for producing hydrogen from water are disclosed herein, it should be noted that any other source for hydrogen known in the art may be substituted for water.

The foregoing discussion discloses and describes the preferred structure and control system for the present invention. However, one skilled in the art will readily recognize from such discussion, and from the accompanying drawings and claims, that various changes, modifications and variations can be made therein without departing from the true spirit and fair scope of the invention.

What is claimed is:

1. An apparatus for transporting hydrogen, comprising:
a hydrogen source;
a carbon dioxide source;
a reactor in communication with said hydrogen source and said carbon dioxide source for causing hydrogen to react with carbon dioxide to form a product selected from the group consisting of a hydrocarbon and an oxygenated hydrocarbon; and
a conduit in communication with said reactor for transporting said product to one of a consumption location or storage location.

2. The apparatus of claim 1, further comprising a conduit in communication with a consumption location for transporting carbon dioxide to one of a reactor location or storage location.

3. The apparatus of claim 1, wherein said reactor is a Sabatier reactor.

4. The apparatus of claim 1, wherein said hydrogen source is water.

5. The apparatus of claim 4, wherein renewable energy from a renewable energy source is used to dissociate water to form hydrogen.

6. The apparatus of claim 1, wherein said carbon dioxide source is a consumption location.

7. The apparatus of claim 1, wherein said product is Methane.

8. The apparatus of claim 1, wherein carbon dioxide from a first consumption location is vented to the atmosphere and said carbon dioxide source may be a second consumption location or a non-consumption location.

9. A method of transporting hydrogen, comprising the steps of:
providing a source of hydrogen;
providing a source of carbon dioxide;
conducting the hydrogen and the carbon dioxide to a reactor;
reacting hydrogen with carbon dioxide to form a product selected from the group comprising a hydrocarbon and an oxygenated hydrocarbon; and
transporting the product to one of a consumption location or storage location.

10. The method of claim 9, further comprising the step of transporting carbon dioxide from a consumption location to one of a reactor location or storage location.

11. The method of claim 10, wherein said hydrogen source is water.

12. The method of claim 11, further comprising the step of using renewable energy from a renewable energy source to dissociate water to form hydrogen.

13. The method of claim 10, further including the steps of venting carbon dioxide to the atmosphere at a first consumption location and providing carbon dioxide from one of a second consumption location or a non-consumption location.

14. An apparatus for storing hydrogen by using carbon dioxide as a storage medium, comprising:
a hydrogen source;
a carbon dioxide source;
a reactor in communication with said hydrogen source and said carbon dioxide source for causing hydrogen to react with carbon dioxide to form a product selected from the group consisting of a hydrocarbon and an oxygenated hydrocarbon; and
a storage device in communication with the reactor for storing the product.

15. The apparatus of claim 14, further comprising a conduit in communication with a consumption location for transporting carbon dioxide to one of a reactor or storage device.

16. The apparatus of claim 14, wherein said reactor is a Sabatier reactor.

17. The apparatus of claim 14, wherein said hydrogen source is a renewable energy source.

18. The apparatus of claim 17, wherein renewable energy from said renewable energy source is used to dissociate water to form hydrogen.

19. The apparatus of claim 18, wherein said carbon dioxide source is a consumption location.

20. The apparatus of claim 14, wherein said product is Methane.

21. A method of storing hydrogen by using carbon dioxide as a storage medium, comprising the steps of:
providing an amount of hydrogen;
providing an amount of carbon dioxide;
conducting the hydrogen and the carbon dioxide to a reactor;
reacting an amount of hydrogen with an amount of carbon dioxide to form a product selected from the group consisting of a hydrocarbon and an oxygenated hydrocarbon; and
storing the product.

22. The method of claim 21, wherein the hydrogen and carbon dioxide are reacted by a Sabatier reactor.

23. The method of claim 21, wherein the hydrogen and carbon dioxide are reacted by a photo-electrolyzing device.
